(19) United States
(12) Patent Application Publication
(10) Pub. No.: US 2005/0234556 A1
(43) Pub. Date: Oct. 20, 2005

(54) Intervertebral disk prosthesis

(76) Inventor: Peter Kretschmer, Bruchkobel (DE)

Correspondence Address: Friedrich Kueffner, Suite 910, 317 Madison Avenue, New York, NY, 10017, US

(21) Appl. No.: 11/102,489

(22) Filed: Apr. 8, 2005

(30) Foreign Application Priority Data
Apr. 8, 2004 (DE) 10 2004 017 506.3

(51) Int. Cl. A61F 2/44

(52) U.S. Cl. 623/17.15; 623/17.13

(57) ABSTRACT

An intervertebral disk prosthesis includes a first anchoring device for anchoring the prosthesis to a first vertebral body and a second anchoring device for anchoring the prostheses to a second vertebral body. The two anchoring devices are moveably coupled to each other. The coupling between the two anchoring devices may include at least one deformable element. The coupling may be constructed as a single piece integrally with the first anchoring device and/or with the second anchoring device.

### BACKGROUND OF THE INVENTION

1. Field of the Invention

The present invention relates to an intervertebral disk prosthesis with a first anchoring device for anchoring the prosthesis at a first vertebral body and a second anchoring device for anchoring the prosthesis at a second vertebral body.

2. Description of the Related Art

Intervertebral disk prostheses of the above-described type are known in the art. The prostheses have the purpose of replacing a natural intervertebral disk. A natural intervertebral disk is particularly distinguished by its flexibility. However, if the intervertebral disk prosthesis replacing the intervertebral disk is rigid, then those vertebral bodies to which the prosthesis is anchored are not moveable relative to each other. This results in an impairment of the mobility of the vertebral column of the patient wearing the prosthesis.

### SUMMARY OF THE INVENTION

Therefore, it is the primary object of the present invention to further develop the intervertebral disk prosthesis of the above described type in such a way that the mobility of the vertebral column is less impaired.

In accordance with the present invention, the above object is met by providing a coupling means for moveably connecting the two anchoring elements.

The present invention is based on the finding that the two vertebral bodies can be moved relative to each other if the two anchoring devices are moveable relative to each other. On the other hand, the two anchoring devices may not be completely separate from each other. The coupling means according to the present invention meets both requirements. On the one hand, the two vertebral bodies involved are still connected to each other to some extent, while on the other hand, they are moveable relative to each other to a certain extent. This reproduces to the greatest extent a natural state of an intervertebral disk between the two vertebral bodies.

In couplings which have coupling elements which engage in each other but are moveable relative to each other there frequently is the problem that the components which engage in each other (for example, ball joints or the like) are jammed relative to each other. Moreover, very narrow manufacturing tolerances have to be maintained.

In accordance with a particularly preferred embodiment of the present invention, the coupling means has at least one deformable element. As a result, coupling elements which engage in each other may not be necessary. Consequently, jamming cannot occur.

In accordance with a particularly simple solution according to the present invention, the deformable element is twistable. Twisting has additional advantages as compared to other deformations, such as stretching and upsetting.

To insure that the coupling can automatically return into its initial position, the present invention provides that the deformable element is preferably elastically deformable.

A particularly simple configuration is achieved if the deformable element is a leaf spring.

In accordance with an additional preferred embodiment, the deformable element is oblong and extends parallel to a principal plane of the first anchoring device.

As a result, it is particularly easily possible to preset the restoring force which counteracts the deformations.

In accordance with another preferred feature of the present invention, the first anchoring element has a window-like opening and the deformable element is formed in the window in the manner of a rung.

This provides the possibility of forming and arranging the deformable element in a particularly space-saving manner.

In accordance with a particularly preferred embodiment of the invention, both anchoring devices each have a deformable element, wherein the deformable elements are coupled to each other.

As a result, in spite of the use of simple deformable elements, it is possible to insure the mobility of the two anchoring devices relative to each other in the sense of two degrees of freedom.

The two deformable elements may be of different configurations. However, in order to avoid that one of the degrees of freedom is preferred, the present invention preferably provides that the two deformable elements are the same.

In accordance with another preferred feature of the invention, those directions in which the two deformable elements are deformable, are different from each other.

This configuration has the particular purpose of realizing a mobility of the two anchoring elements relative to each other in the sense of different degrees of freedom.

In accordance with a preferred feature, the directions of the formation of the two deformable elements extend perpendicularly relative to each other.

In accordance with a particularly simple preferred embodiment of the invention, the two deformable elements are twistable; specifically, the elements are twistable about axes which extend perpendicularly to each other.

As a result of this configuration of the present invention, the two principal planes of the anchoring devices can be tilted about any desired axes parallel to the principal planes, just as is the case in a natural intervertebral disk.

In accordance with a particularly preferred feature providing support, the two torsional axes are located parallel to the principal planes of the two anchoring elements.

Finally, in accordance with a preferred feature of the present invention, the coupling means is constructed integrally as a single piece with the first anchoring device as such or with the second anchoring device. This configuration is particularly advantageous with respect to the manufactured, implantation and service life of the prosthesis.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a side view of a particularly preferred embodiment of the intervertebral disk prosthesis according to the present invention;

FIG. 2 is a side view of the prosthesis of FIG. 1, but seen from the opposite side;

FIG. 3 is a top view of the prosthesis of FIG. 1;

FIG. 4 is perspective view of the prosthesis of FIG. 1;

### DETAILED DESCRIPTION OF THE INVENTION

The intervertebral disk prosthesis illustrated in the drawing includes two anchoring devices which in the following will be called rings 10 and 12 and which are provided with teeth for entering into one of the adjacent vertebral bodies, not shown. As an example, the teeth are denoted by reference numerals 14 and 16. In the particularly preferred embodiment, the two rings 10 and 12 have an oval contour. Of course, the present invention is not limited to a certain oval contour. Rather, other shapes may be available, such as rectangular, circular, kidney-shaped.

Provided in the rings 10 and 12 are leaf springs 18 and 20, respectively, which are constructed in the manner of a rung in a window. The two leaf springs 18 and 20 are connected to each other in the middle thereof, for example, in the connection area 22.

The two rings 10 and 12, the two leaf springs 18 and 20 as well as the connection area 22 are all constructed as a single piece with each other. This is because the intervertebral disk prosthesis illustrated in the drawing is manufactured from a blank by milling cutting.

When the two rings 10 and 12 are tilted relative to each other so that their principal planes, which in the undeformed state extend parallel to each other, form an acute angle with each other, at least one of the leaf springs 18 and 20 is twisted to the corresponding extent. When such twisting occurs, the two leaf springs 18 and 20 produce a corresponding elastic restoring force which serves to return to the state of parallel principal planes as soon as the reason for tilting of the two rings 10 and 12 relative to each other is no longer there.

The two rings 10 and 12 are only tiltable relative to each other. The rings are particularly not slideable relative to each other laterally and they are not compressible. Consequently, those vertebral bodies at which the two rings 10 and 12 are anchored, can be connected moveably to each other, however, are secured relative to each other, so that they do not move, for example, laterally or in the direction of the axes of the vertebral column.

It should be expressly mentioned that the two leaf springs 18 and 20 do not have to intersect at a right angle. Other angles are possible and are selected in accordance with given circumstances. They also do not have to be straight, but rather may be arched or S-shaped.

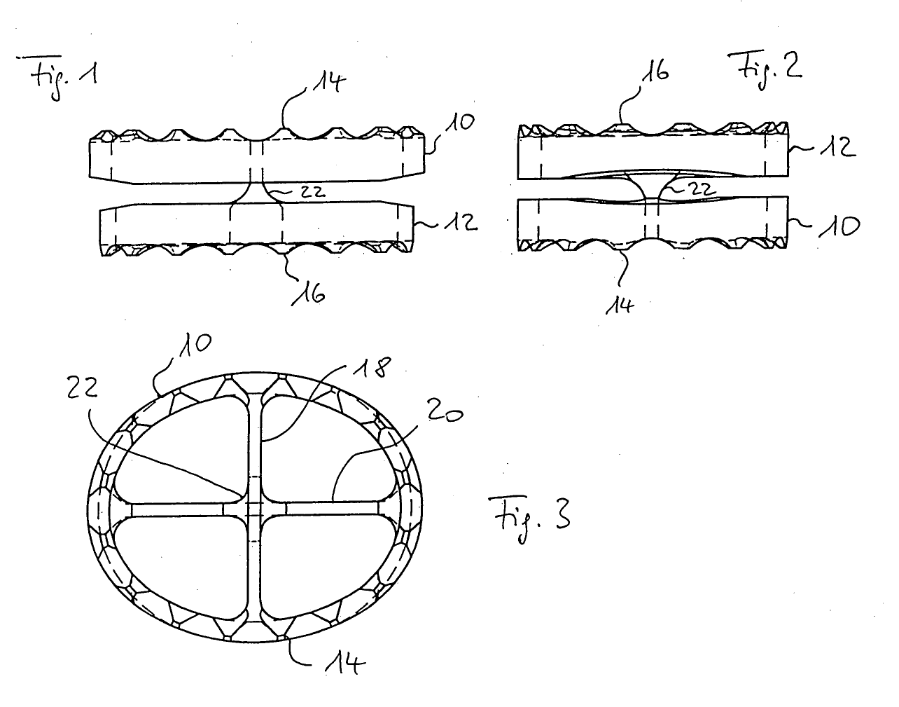

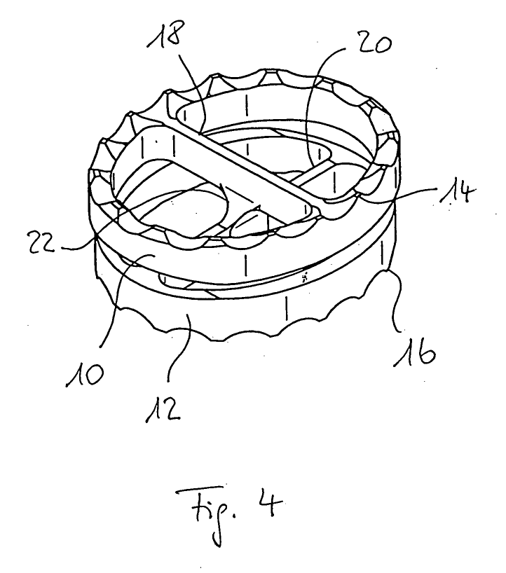

Although not shown in the drawing, the respective openings seen in FIG. 3 to the right and left and above and below the two leaf springs 18 and 20 may be covered, so that no bone substance grows into the openings. The same is true for the space between the two rings 10 and 12 which is particularly visible in FIGS. 1 and 2.

The features of the invention described above and disclosed in the claims as well as the drawing may be significant either individually or in any combination for realizing the various embodiments of the invention.

While specific embodiments of the invention have been shown and described in detail to illustrate the inventive principles, it will be understood that the invention may be embodied otherwise without departing from such principles.

1. An intervertebral disk prosthesis comprising
a first anchoring device for anchoring the prosthesis to a first vertebral body,
a second anchoring device for anchoring the prosthesis to a second vertebral body; and
a coupling means for moveably coupling the two anchoring devices to each other.

2. The prosthesis according to claim 1, wherein the coupling means comprises at least one deformable element.

3. The prosthesis according to claim 2, wherein the deformable element is twistable.

4. The prosthesis according to claim 2, wherein the deformable element is elastically deformable.

5. The prosthesis according to claim 2, wherein the deformable element is a leaf spring.

6. The prosthesis according to claim 2, wherein the deformable element is oblong and extends parallel to a principal plane of the first anchoring device.

7. The prosthesis according to claim 2, wherein the first anchoring device has a window-like opening, and wherein the deformable element is mounted in the opening in the manner of a rung.

8. The prosthesis according to claim 2, wherein the first and second anchoring devices each have a deformable element, wherein the deformable elements are coupled to each other.

9. The prosthesis according to claim 8, wherein the two deformable elements are identical.

10. The prosthesis according to claim 8, wherein directions in which the two deformable elements are different from each other.

11. The prosthesis according to claim 10, wherein the deformation directions of the two deformable elements extend perpendicularly to each other.

12. The prosthesis according to claim 10, wherein the two deformable elements are twistable about axes which extend perpendicularly to each other.

13. The prosthesis according to claim 12, wherein the two axes extend parallel to the principal planes of the two anchoring devices.

14. The prosthesis according to claim 1, wherein the coupling means is integrally constructed as a single piece at least with one of the first anchoring device and the second anchoring device.
